(19) United States
(12) Patent Application Publication
(10) Pub. No.: US 2023/0274834 A1
(43) Pub. Date: Aug. 31, 2023

(54) MODEL-BASED EVALUATION OF ASSESSMENT QUESTIONS, ASSESSMENT ANSWERS, AND PATIENT DATA TO DETECT CONDITIONS

(71) Applicant: Spora Health, Inc., San Francisco, CA (US)

(72) Inventors: Waco Shane Holve, Boise, ID (US); Danny Maurice Miller, San Francisco, CA (US); Terrell David Smith, Palmyra, VA (US)

(21) Appl. No.: 18/017,482

(22) PCT Filed: Jul. 22, 2021
(86) PCT No.: PCT/US21/42847

§ 371 Date: Jan. 23, 2023

Related U.S. Application Data
(60) Provisional application No. 63/055,164, filed on Jul. 22, 2020.
(60) Provisional application No. 63/073,759, filed on Sep. 2, 2020.

(51) Int. Cl. G16H 50/20; G16H 10/60; G16H 10/20

(52) U.S. Cl. CPC G16H 50/20; G16H 10/60; G16H 10/20

(57) ABSTRACT

A software and/or hardware condition detection system for detecting the probability of a particular condition, such as a particular disease or disorder, and identifying opportunities for altering those probabilities is provided. The condition detection system trains one or more machine learning models to generate condition probabilities for patients using training data collected from any number of sources. The condition probability system then surveys patients and/or their healthcare providers for information about the patient via, for example, a questionnaire, and applies one or more trained models to the collected patient information to detect conditions for the patient. Additionally, the condition detection system simulates different answers to the survey for the patient, generates condition probabilities for those simulated answers, and compares those generated condition probabilities to the patient's current condition probability. Through these comparisons, the condition detection system can identify opportunities to change one or more of the patient's condition probabilities.

### CROSS-REFERENCE TO RELATED APPLICATIONS

This application claims the benefit of U.S. Provisional Patent Application No. 63/055,164, titled “Disease Detection System,” filed on Jul. 22, 2020, which is herein incorporated by reference in its entirety. This application further claims the benefit of U.S. Provisional Patent Application No. 63/073,759, titled “Disease Detection System,” filed on Sep. 2, 2020, which is hereby incorporated by reference in its entirety.

### BACKGROUND

Providing medical treatment and health care for patients with one or more conditions requiring repeated treatment is a major issue. Early detection and diagnosis of various conditions, such as diseases, enables patients and medical providers to begin treatment plans sooner, which often results in better patient outcomes. Patients whose conditions are detected early are also better positioned to make important decisions for themselves regarding various matters, such as care and support decisions, financial matters, legal matters, and so on. Additionally, an early diagnosis can make patients eligible for certain clinical trials, which can advance research and provide medical benefits. Repeated visits, diagnosis, treatment, therapy, etc. is a shared responsibility between medical workers, the patient, and often others (e.g., family), with the patient performing some actions on their own to provide treatment, medical workers periodically checking up on the patient to ensure that the patient is following a treatment plan and to determine whether the treatment plan is working, and others performing various support roles.

Many organizations collect information, such as health information, about individuals. For example, the National Health and Nutrition Examination Survey (NHANES) program conducted by the National Center for Health Statistics (NCHS) assesses the health and nutritional status of individuals in the United States. The NHANES includes a database containing health records for individuals that includes over 7,000 variables that can have associated values. As another example, the Behavioral Risk Factor Surveillance System (BRFSS) maintains a database containing health records for individuals that includes over 600 variables and associated values. These data values may be provided by individuals via physical examinations, laboratory tests, interviews, questionnaires, surveys, and so on. Such questions may include, for example, “In the last 30 days how many frozen pizzas have you eaten?,” “In your immediate family, do you have any history of diabetes?,” “Has a doctor ever told you that you are overweight?,” “Do you get shortness of breath walking up hill or a flight of stairs?,” “Have you ever been told you had an anxiety disorder?,” “How often do you have trouble sleeping?,” “Does arthritis affect whether you work?,” “How often do you eat french fries or fried potatoes?,” etc. The variables and corresponding values for an individual may be linked together by a Health Insurance Portability and Accountability Act-compliant unique number generated by, for example, BRFSS.

### BRIEF DESCRIPTION OF THE DRAWINGS

FIG. 1 is a block diagram illustrating an environment in which the condition detection system operates.

FIG. 2 is a flow diagram illustrating the processing of a condition detection component.

FIG. 3 is a block diagram illustrating the processing of a feature selection component.

FIG. 4 is a flow diagram illustrating the processing of a model selection component.

FIG. 5 is a flow diagram illustrating the processing of a generate condition probability groups component.

FIG. 6 is a flow diagram illustrating the processing of a simulate component.

FIG. 7 is a flow diagram illustrating the processing of a build group representative component.

FIG. 8 is a flow diagram illustrating the processing of an identify opportunities component.

### DETAILED DESCRIPTION

The inventors have recognized that conventional approaches to detecting conditions within patients have significant disadvantages. For example, typical condition detection techniques often rely on intrusive or lengthy medical tests, such as biopsies or tests that require lab testing and results. In these cases, a patient may be reluctant to seek the necessary testing and/or suffer from lengthy delays in obtaining results. These delays can hinder the patient's ability to obtain timely treatment or may put the patient in a position to require additional, more expensive treatments. Furthermore, typical detection systems do not identify opportunities for changing the condition or condition probability. Further, many detection systems simply provide detection results during or in response to a single patient visit, without providing updated results in response to changes in underlying comparison data. Moreover, some detection systems rely on records from a single source due to problems related to standardization of data between sources. The inventors have determined that a condition detection system that addresses these problems would have great value to patients and healthcare providers.

Accordingly, the inventors have conceived a software and/or hardware condition detection system for detecting the probability of one or more conditions, such as a particular disease, disorder, syndrome, etc., and identifying opportunities for altering those probabilities. In some embodiments, the condition detection system trains one or more machine learning models to generate condition probabilities for targets (e.g., target individuals), such as users or patients, using records collected from any number of sources as training data and, in some cases, any number of transformations or augmentations of the data, such as normalizing (e.g., calculating a statistical standard score, t-score, z-score, etc. for each value collected for a particular variable), scaling, applying mathematical transforms (e.g., Laplace transform, etc.), and so on. Thus, the condition detection system can dynamically generate new variables for features, rather than relying on static underlying data, that may be more predictive than features found using only the underlying data. Thus, the condition detection system addresses problems of static underlying features found in other detection systems. The condition probability system then surveys patients for information about themselves via, for example, a questionnaire, and applies the trained model or models to the collected patient information to generate condition probabilities for the patient. Furthermore, the condition detection system simulates different survey answers for the patient, generates condition probabilities for those simulated answers, and compares those generated condition probabilities to the patient's current condition probability (i.e., baseline). Through these comparisons, the condition detection system can identify opportunities to change the patient's condition probabilities by identifying which changes to which answers will have the greatest (or least) effect on any one or more of the patient's condition probabilities. Moreover, because the condition detection system can detect conditions within patients using survey results, the condition detection system can quickly provide updates to the patient and the patient's healthcare providers without lengthy and expensive procedures, thereby conserving valuable resources for both the patient, the healthcare provider, and the healthcare system.

In some embodiments, a model-based condition detection system analyzes records, such as health records, for a number of individuals and constructs probability models based on those records, with each probability model determining the probability that a particular condition exists within a patient or that the patient will acquire the condition. For example, one probability model (or set of models) may be used to determine the probability of a person having diabetes while another probability model (or set of models) is used to determine whether a person has or will acquire pulmonary hypertension. Once the models are generated, the condition detection system can apply the models to patient data to determine the probability the patient has (or may acquire) a particular condition. For example, the patient may respond to a health assessment represented as data structures or documents (e.g., electronic documents) representing questions of a survey or questionnaire with a set of answers. These answers can be provided to the models to predict whether the patient has a corresponding condition. Furthermore, the condition detection system can simulate different answers to those questions to find one or more hypothetical sets of answers to the questions that would result in a different probability and present those results to the patient in the form of opportunities for the patient to change their probability (in some cases under the supervision of a physician or other health care provider), such as a recommendation to change eating and/or exercise habits, prescription drugs, weight, etc. In this manner, the condition detection system provides improved methods and systems for assessing questions, answers, and patient data to determine probabilities related to one or more conditions and highlight potential opportunities to change these probabilities. These identified opportunities, in turn, can trigger the creation of a new treatment or patient care plan or the modification of an existing plan, which may provide the patient with better outcomes and health and with quicker responses to changes in the patient's condition, which can conserve resources of the patient and the medical field.

In some embodiments, the condition detection system receives an indication of a condition to be predicted (i.e., determining the probability that a patient has or will acquire the condition). A condition can be selected based on having a variable that corresponds to an explicit question directed to whether an individual has the condition (e.g., “Have you been diagnosed with coronary artery disease?” or “Has a doctor ever told you that you have diabetes?”). In some cases, a condition can be selected based on whether multiple variables can be used to infer whether an individual has the condition. For example, variables relating to Body Mass Index (BMI) and waist circumference may indicate whether an individual is obese. To be effective at predicting a condition, a threshold number of records (e.g., 3,000) may be needed as positive examples of individuals with the condition. The threshold number can vary based on the type of condition.

As discussed above, health records for different individuals (typically anonymized to protect the identity of the underlying individuals) can be obtained from any number of sources. Moreover, the underlying variables and associated values can be both continuous (e.g., height) or categorical (e.g., a Y/N question). In some embodiments, the condition detection system uses variables that can be measured by an individual (e.g., using a tape measure) and relate to general health knowledge questions (e.g., relating to family history). In some cases, the condition detection system excludes variables such as those relating to laboratory results and blood pressure readings. The condition detection system identifies, from among the available variables, those variables that are predictive (i.e., effective at identifying that an individual has a corresponding condition) and uses a combination of those predictive variables as features to create predictive models using machine learning techniques. In some embodiments, the condition detection system employs a data mining process to initially identify a subset of the variables of the variable set that tend to be predictive of the condition. For example, the data mining process may identify 80 of the thousands of variables as predictive variables. Once the predictive variables are identified, the condition detection system can eliminate either predictive variables that are closely related to the identified condition (removing forward looking bias) or spurious predictive variables (e.g., determined to have no relevance to the condition). For example, a question relating to whether an individual is taking insulin is not useful in predicting whether the individual has prediabetes because the two are closely related. In some cases, the condition detection system eliminates predictive variables that do not have at least a threshold percentage (between 0% and 100%) of the records with a positive or negative indicator for the identified condition. For example, if only 49% of the records have answers to whether the individual has prediabetes or have a value for a certain predictive variable, the condition detection system can eliminate that predictive variable. The remaining predictive variables (e.g., 30 of the 80) are considered candidates for features of the training data used to train machine learning models for generating condition probabilities.

In some embodiments, the condition detection system determines whether a variable is predictive by assigning a predictive score to each variable by testing its ability to predict the target variable or variables corresponding to the condition (e.g., prediabetes, obesity). The predictive score can be generated based on a person's demographics (e.g., age, sex at birth, ethnicity, BMI). Based on this information, the condition detection system identifies instances of patient data that have all demographics provided and then fits a naïve model (e.g., a gaussian naïve bayes model, a decision tree model, and so on) to the demographic data to assess how predictive the demographic data is of the condition (i.e., determine a predictive value for the demographics data). Subsequently, each potential predictive variable (i.e., the candidates considered for features of the training data discussed above) is appended to the demographic data to create composite data, and the condition detection system fits a new model to the composite data. The condition detection system then fits the naïve model to the data (i.e., the demographic data and appended variable values) to determine how predictive this composite data is of the condition (i.e., determine a predictive value for the composite data). The condition detection system then compares the fit of the naïve model to the demographic data to the fit of the naïve model to the composite data to generate a difference, or delta, between the two. The delta is considered the “information gain.” The condition detection system then deems any variable that has positive information gain above a predetermined threshold (e.g., 1%, 5%, 20%) to be a predictive variable. In some embodiments, the condition detection system identifies predictive variables by generating predictive power scores for each using a generic tree-based model. Predictive power scores are different from correlation in the sense that, instead of looking at only the correlation, the predictive power scores break down linear and non-linear patterns within the data and allow the condition detection system to systematically eliminate a large majority of the variables (e.g., relating to an appendectomy) that are not predictive, leaving only predictive variables. The predictive power scoring system fits naïve models to a variable and compares the variable to the target variable to see what can be learned from the relationship. The Predictive Power Score system is further described at https://pypi.org/project/ppscore/, https://github.com/8080labs/ppscore/#calculation-of-the-pps, and https://towardsdatascience.com/rip-correlation-introducing-the-predictive-power-score-3d90808b9598, each of which is herein incorporated by reference in its entirety.

In some cases, values for variables may be missing. For example, a patient may have chosen not to answer a particular question in a survey, may never have been tested or measured for a particular attribute or condition, or may never have been presented with a corresponding question. In order to resolve these discrepancies, the condition detection system may fill in values for predictive variables in records with missing values. For example, for categorical variables (e.g., Y/N or scale of 1-10), the condition detection system can fill in missing data with a “refused to answer” value. In addition, if a variable is found to be highly predictive of the condition and if a question relating to that variable is not answered, the condition detection system may assume “no” is a fair response (e.g. “Have you been diagnosed with hypertension?”). For continuous variables (e.g., weight), the condition system may fill in missing data with an average (e.g., mean, median, mode) value of that variable.

Once the predictive variables are identified, the condition detection system attempts to fit a Generalized Linear Model (GLM) to subsets of the predictive variables to identify subsets that are effective at predicting the condition. Depending on the number of predictive variables and the desired number of features, the condition detection system may fit the GLM to each possible combination of N predictive variables. For example, if there are 30 predictive variables and 25 features to be selected, the condition detection system fits the GLM to each combination of 25 predictive variables. As another example, the condition detection system may fit the GLM to every possible combination of the predictive variables or every possible combination of at least a threshold number of predictive variables, where the threshold is determined by a user or automatically by the condition detection system as a percentage of the number of predictive variables, randomly, and so on. As another example, the condition detection system may randomly generate combinations of predictive variables and fit the GLM to the randomly selected combinations.

In some embodiments, the condition detection system evaluates the accuracy of the GLM, for example, based on analysis of type I and type II errors. Type I errors occur when a true null hypothesis is rejected (i.e., a false positive), such as when the GLM predicts that a patient who does not have the condition has the condition. Type II errors occur when a true null hypothesis is not rejected (i.e., a false negative), such as when a model predicts that a patient who has the condition does not have the condition. If no combinations are found to have sufficient accuracy, the condition detection system may evaluate combinations of fewer (e.g., N−2) and/or more (N+2) predictive variables. If multiple combinations are found to have sufficient accuracy, the condition detection system may take the union of the variables in those combinations as the features. Alternatively, the condition detection system may evaluate each combination as separate features used in training multiple models. The condition detection system may also generate plots to assist in a manual selection of features. For example, if weight is a variable, a plot may have an x-axis of weight ranges and a y-axis indicating the percent of records having that condition for each weight range.

Given a collection of possible models (and corresponding sets of features) for predicting a condition within a patient, the condition detection system determines which models have sufficient predictive capability to generate accurate predictions. To determine if a model has sufficient capability, the condition detection system trains models using the training data (e.g., data collected from NHANES, BRFSS, or other collections of data) and evaluates the predictive ability of each model, for example, based on type I and type II errors. For example, the condition detection may determine that any model having an accuracy above a predetermined threshold (e.g., 70%, 85%, 90%, 95%) has sufficient predictive capability. In another example, the condition detection system may select a threshold number or percentage of models analyzed (e.g., top 10, top 10%, and so on). After a number of models are identified as having sufficient predictive capability, the condition detection system may employ an exhaustive process to train and evaluate the predictive capabilities of each possible combination (or ensemble) of the models. For example, if n models were identified, the condition detection system may evaluate, for example, nCr combinations (i.e., (n!)/(r!(n−r)!)) where r represents a number of models to be selected (in some examples, the condition detection system may evaluate nCr combinations for multiple values of n and/or r. In some embodiments, a model will not be accepted into the ensemble unless the model, when added to the ensemble, improves the performance of the ensemble. A model is assumed to provide value if the accuracy of the combination does not decrease and if the type I and type II statistical errors decrease. If, however, the accuracy decreases but the type I and type II errors decrease more than the accuracy, the model may be determined to have value.

If one model is selected, the condition detection system can use the selected model as the condition probability model or condition detection model (i.e., the model used to generate condition probabilities for patients for the corresponding condition). If multiple models are selected, the condition detection system can generate weights for the models to produce a single ensemble of models to be used as the condition detection model. In some embodiments, the condition detection system initially assigns equal weight to the models and then applies, for example, a hyper parameterization process, such as an evolution optimization, to determine what allocation of weights leads to the most accurate model to prevent selection bias for the absolutely best model. Once the weights are finalized, the model can be saved and can be deployed to production. One of ordinary skill in the art will recognize that the disclosed technology may operate with any form of classification models (or classifiers), such as Gaussian models, boosting models, neural networks (e.g., fully connected, convolutional, recurrent, autoencoder, restricted Boltzmann machine), support vector machines, Bayesian classifiers, k-means classifiers, and so on. The ensemble may be combined using a voting classifier. When the classifier is a deep neural network, the training results in a set of weights for the activation functions of the deep neural network. A support vector machine operates by finding a hyper-surface in the space of possible inputs. The hyper-surface attempts to split the positive examples from the negative examples by maximizing the distance between the nearest of the positive and negative examples to the hyper-surface. This step allows for correct classification of data that is similar to but not identical to the training data. Various techniques can be used to train a support vector machine. In some cases, the component may employ adaptive boosting. Adaptive boosting is an iterative process that runs multiple tests on a collection of training data. Adaptive boosting transforms a weak learning algorithm (an algorithm that performs at a level only slightly better than chance) into a strong learning algorithm (an algorithm that displays a low error rate). The weak learning algorithm is run on different subsets of the training data. The algorithm concentrates more and more on those examples in which its predecessors tended to show mistakes. The algorithm corrects the errors made by earlier weak learners. The algorithm is adaptive because it adjusts to the error rates of its predecessors. Adaptive boosting combines rough and moderately inaccurate rules of thumb to create a high-performance algorithm. Adaptive boosting combines the results of each separately run test into a single, very accurate classifier. Adaptive boosting may use weak classifiers that are single-split trees with only two leaf nodes.

Once the condition detection model has been generated, the condition detection system can apply it to patient data (e.g., survey answers) to predict the probability that the patient has the condition for which the model was trained. In some examples, the condition detection system presents a patient with a survey or questionnaire and asks the patient to provide answers for each of a number of questions, each question relating to one of the variables used to train the model. In some examples, the condition detection system may receive patient data through a survey or data collection process performed by a third party. The condition detection system applies the model to the patient's answers to generate a baseline prediction for the condition (e.g., the patient has the condition or does not have the condition). In this manner, the patient's current state relative to the condition can be assessed. Accordingly, if the patient is predicted to have the condition, additional tests can be scheduled, and the patient can begin any appropriate treatment plans. Thus, the condition detection system can provide the patient with an improved method that relies on survey answers from the patient and without intrusive tests for early detection of conditions.

Furthermore, the condition detection system can simulate different sets of patient data for the patient (i.e., with different hypothetical answers to survey questions) and use condition detection models to generate condition probabilities for each simulated set. Some of the questions will have a wide variety of potential answers that may change for a particular patient over time (e.g., “What is your annual household income?”). These questions and answers are referred to as “flex questions” and “flex answers.” Other questions have answers that typically do not substantially change for a user over time or once they have reached a certain age (e.g., “What is your standing height?”). These questions and answers may be referred to as “non-flex questions” and “non-flex answers.” The condition detection system identifies ranges of flex answers to each of the flex questions and then generates every combination of those flex answers with the identified range. For example, if a question (e.g., “How much do you weigh?” or “What is your waist size?”) has a range of answers, the condition detection system determines all potential answers for that patient for the question. The condition detection system does this for every flex question in order to generate possible combinations of survey answers for the patient. In some examples, the condition detection system attempts to generate every conceivable set of responses that the patient may provide to the survey. Accordingly, there may be any number of different combinations generated. In all combinations, the non-flex answers are the same for a particular non-flex question and a corresponding patient.

For each combination of possible answers, the condition detection system applies the condition detection model to the combination of possible answers to determine a condition probability for that combination. The total number of outputted condition probabilities equals the total number of combinations that the condition detection model is applied to. The combinations may then be grouped into, for example, equally sized groups of answer combinations, such as quartiles, based on their condition probabilities. For example, one “condition probability group” may have probabilities 0 to 0.3, another 0.3 to 0.53, etc. Then, for each flex question, the average of the answers in each condition probability group is computed to build, for each condition probability group, a hypothetical representative or average individual for the condition probability group. For example, if ten condition probability groups each represent three million combinations, the average answer to a weight question for each condition probability group would be the sum of the weights in each group divided by three million.

The condition detection system uses these condition probability groups to help determine how changing values to the flex answers can impact the patient's probability of having or acquiring the condition. Moreover, the questions, answers, and condition probabilities can be displayed in an easy to read condition probability table, with both baseline answers and average flex answers for the patient, giving the patient and the patient's healthcare providers an easy to use chart for identifying potential changes to alter any one of their condition probabilities. In some embodiments, the condition detection system receives a target condition probability from a patient (e.g., 0.25, “0.15 less than my current baseline,” “0.4 above my baseline”). In response, the condition detection system identifies which probability group the target condition probability falls into and provides the average answers for that group (i.e., the hypothetical representative or average individual). For example, if a patient wants to determine how to reduce their risk for diabetes from 0.8 to 0.4, the condition detection system outputs the average of the answers in the group that includes the probability 0.4 (e.g., 0.3 to 0.53). The outputted answers may allow the patient to determine which variable values the patient can or should adjust (i.e., which questions the patient can work to change their answers for) to achieve the target condition probability. The outputted answers may also enable healthcare providers to quickly and easily understand which answers need to be adjusted in order to lower the risk factor for the patient. Thus, the condition detection system provides patients and healthcare providers with an improved system for detecting conditions within patients, which can lead to earlier detection, reduced medical costs, and better long-term and short-term outcomes for patients.

TABLE 1

| | DRQSDIET | BMXBMI | BMXHT | BMXWT | BPQ100D | DBD910 | FSQ165 | HSD010 | MCQ300C | RXDCOUNT |
|---|---|---|---|---|---|---|---|---|---|---|
| baseline | 2 | 28.8 | 172.4 | 85.6 | 1 | 0 | 2 | 2 | 1 | 2 |
| 10.0% | 1.5 | 28.8 | 172.4 | 85.6 | 1.3 | 2.5 | 2 | 2.7 | 1 | 3.7 |
| 20.0% | 1.5 | 28.8 | 172.4 | 85.5 | 1.5 | 2.5 | 2 | 2.6 | 1 | 3.4 |
| 30.0% | 1.5 | 28.8 | 172.4 | 85.5 | 1.6 | 2.5 | 2 | 2.6 | 1 | 3 |
| 40.0% | 1.5 | 28.7 | 172.4 | 85.4 | 1.5 | 2.5 | 2 | 2.6 | 1 | 2.4 |
| 50.0% | 1.5 | 28.8 | 172.4 | 85.6 | 1.5 | 2.5 | 2 | 2.6 | 1 | 1.7 |
| 60.0% | 1.5 | 28.8 | 172.4 | 85.7 | 1.5 | 2.5 | 2 | 2.5 | 1 | 1.3 |
| 70.0% | 1.5 | 28.8 | 172.4 | 85.6 | 1.5 | 2.5 | 2 | 2.4 | 1 | 1 |
| 80.0% | 1.5 | 28.8 | 172.4 | 85.7 | 1.6 | 2.5 | 2 | 2.3 | 1 | 0.8 |
| 90.0% | 1.5 | 28.8 | 172.4 | 85.6 | 1.7 | 2.5 | 2 | 2.2 | 1 | 0.6 |
| 100.0% | 1.5 | 28.9 | 172.4 | 85.9 | 1.9 | 2.5 | 2 | 1.8 | 1 | 0.3 |

| | WHD050 | WHD110 | WHD120 | WHD140 | DMDHHSIZ | RIAGENDR | RIDAGEYR | RIDRETH1 | redFatCal | hhincome |
|---|---|---|---|---|---|---|---|---|---|---|
| baseline | 188 | 200 | 157 | 210 | 2 | 1 | 58 | 3 | 2 | 15 |
| 10.0% | 161.4 | 200.6 | 157 | 212.6 | 2 | 1 | 60.6 | 3 | 1.4 | 15 |
| 20.0% | 166.2 | 200.4 | 157 | 212.2 | 2 | 1 | 60.6 | 3 | 1.5 | 15 |
| 30.0% | 167.8 | 200.1 | 157 | 211.9 | 2 | 1 | 60.5 | 3 | 1.5 | 15 |
| 40.0% | 167.7 | 200.2 | 157 | 211.8 | 2 | 1 | 60.5 | 3 | 1.5 | 15 |
| 50.0% | 167.1 | 200.2 | 157 | 211.9 | 2 | 1 | 60.5 | 3 | 1.5 | 15 |
| 60.0% | 168.5 | 200.1 | 157 | 211.7 | 2 | 1 | 60.5 | 3 | 1.5 | 15 |
| 70.0% | 169.7 | 199.8 | 157 | 211.4 | 2 | 1 | 60.5 | 3 | 1.5 | 15 |
| 80.0% | 170.7 | 199.6 | 157 | 211.1 | 2 | 1 | 60.4 | 3 | 1.6 | 15 |
| 90.0% | 172.6 | 199.2 | 157 | 210.5 | 2 | 1 | 60.4 | 3 | 1.6 | 15 |
| 100.0% | 176.8 | 198 | 157 | 208.7 | 2 | 1 | 60.1 | 3 | 1.7 | 15 |

| | mostDelta | mostBMI | tenDelta | tenBMI | oneDelta | oneBMI | mostOneDelta | tenOneDelta | probability |
|---|---|---|---|---|---|---|---|---|---|
| baseline | 21.3 | 32 | 11.3 | 30.5 | −0.7 | 28.7 | 22 | 12 | 0.7776762 |
| 10.0% | 23.9 | 32.4 | 11.9 | 30.6 | −27.3 | 24.6 | 51.1 | 39.2 | 0.508557 |
| 20.0% | 23.8 | 32.4 | 11.9 | 30.6 | −22.2 | 25.4 | 46 | 34.1 | 0.5834419 |
| 30.0% | 23.5 | 32.3 | 11.7 | 30.5 | −20.6 | 25.6 | 44.1 | 32.3 | 0.6267478 |
| 40.0% | 23.5 | 32.3 | 11.9 | 30.5 | −20.6 | 25.6 | 44.1 | 32.4 | 0.7036237 |
| 50.0% | 23.2 | 32.3 | 11.4 | 30.6 | −21.6 | 25.5 | 44.8 | 33.1 | 0.728643 |
| 60.0% | 22.8 | 32.3 | 11.1 | 30.5 | −20.5 | 25.7 | 43.3 | 31.6 | 0.8098923 |
| 70.0% | 22.6 | 32.3 | 11 | 30.5 | −19.1 | 25.9 | 41.7 | 30.1 | 0.8102111 |
| 80.0% | 22.2 | 32.2 | 10.7 | 30.5 | −18.1 | 26.1 | 40.3 | 28.8 | 0.8500581 |
| 90.0% | 21.8 | 32.1 | 10.5 | 30.4 | −16.2 | 26.3 | 38 | 26.6 | 0.8575988 |
| 100.0% | 19.5 | 31.9 | 8.7 | 30.2 | −12.5 | 27 | 32 | 21.3 | 0.8941889 |

Table 1 illustrates a sample condition probability table in accordance with some embodiments of the disclosed technology. The leftmost column includes labels for the baseline combination of answers and ten generated condition probability groups (e.g., “baseline,” “10.0%,”, etc.). The next 28 columns of the table represent the variables (discussed in further detail below with respect to Table 2) that were used to train a condition detection model (e.g., models of an ensemble model) and the corresponding survey questions. The top, or “baseline,” row of the table contains the patient's current baseline answers to the questions (what the patient answered on the survey). Subsequent rows represent each condition probability group and include the condition probability group's average answers to the questions. In this example, ten equally spaced groups (deciles) each contain 10% of the number of combinations of possible flex answers. Each percentage value (leftmost column) represents the percent of combinations in a group with condition probabilities up to the values in the probability column (rightmost). For example, 10% of the combinations have a condition probability up to 0.508577 while 60% of the combinations have a condition probability up to 0.8098923. The column BMXHT (standing height) has all the same answers as that of its baseline and is an example of a non-flex question with a non-flex answer. The column WHD050 (“How much did you weigh a year ago?”) has different answers from that of its baseline and is an example of a flex question with flex answers. In some cases, a column may have answers with very small differences between one another because, for example, the decimal places are not expanded out. For example, BMXBMI has answers such as 28.8, 28.6, and 28.7 because the decimal is rounded to the tenths place. The answer is intentionally rounded and may indicate the question is less material of a data point to a person's overall probability for the condition. One of ordinary skill in the art will recognize that while Table 1 is provided as an example, the condition probability system may generate condition probability charts using any number of variables or questions, any number of groups (e.g., five, 50, 100), and so on.

From the condition probability table of Table 1, one can determine the patient's answers needed to achieve a target condition probability by first determining the group that the target condition probability lies in and then reading the average answers from the corresponding row. For example, a target condition probability of 0.55 would lie between 0.508557 and 0.583442 and fall in the 0.583442 probability group (i.e., the 20% row). Then, examining the average answers in the row for that group, the patient would need, for example, a redFatCal of 1.5, oneDelta of −22.2, and so on. Thus, the condition probability table allows a patient and/or their healthcare provider(s) to quickly compare the patient's current baseline to a target condition probability group to identify changes that the patient can make (potentially under supervision of a medical professional) to get closer to the patient's target condition probability. The condition detection system may also include in the condition probability table an indication of whether each variable is negatively or positively correlated with the condition by, for example, including positive and negative signs, shading or coloring the variables, and so on.

In some embodiments, the condition detection system identifies opportunities for the patient to achieve their target condition probability. For example, the condition probability tables may include an indication of how far the patient's current baseline answers are from the average answers of the target condition probability group, such as a table highlighted with different colors based on the number of standard deviations the patient's answer to a particular question is from the average value for a condition probability group (and depending on whether the corresponding variable is negatively or positively correlated with the condition). As another example, the condition probability system may track changes in individual patient data and corresponding probabilities overtime to determine, for example, which changes lead to the greatest (or smallest) changes in condition probability over time, which variable values patients have been most (or least) successful in changing, and so on. Moreover, the condition detection system may feed this information back into the training data as a basis for enhancing and improving the accuracy of condition detection models over time, through different training stages for one or more models. As another example, in addition to building a group representative for each condition probability group based on average flex answers, the condition detection system may normalize those values based on the underlying data and show the patient's baseline distance from each so the patient and/or the patient's healthcare provider can better understand which variables the patient is closest and/or furthest away from achieving. In this manner, the patient and/or the patients healthcare provider can optimize resources in attaining a desired or target condition probability, thereby conserving valuable resources (e.g., time and money) and providing for better patient outcomes.

TABLE 2

| Name | Description |
|---|---|
| oneDelta | Difference between what the patient weighed 1 year ago |
| | and now |
| DRQSDIET | Are you currently on any kind of diet, either to lose weight |
| | or for some other health-related reason? |
| BMXBMI | Body Mass Index (kg/m**2) |
| BMXHT | Standing Height (cm) |
| BMXWT | Weight (kg) |
| RIAGENDR | Gender |
| RIDAGEYR | Age in years at screening |
| RIDRETH1 | Ethnicity - Recode |
| BPQ100D | (Are you/Is SP) now following this advice to take |
| | prescribed medicine? |
| HSD010 | {First/Next} I have some general questions about |
| | {your/SP's} health. Would you say {your/SP's} health in |
| | general is . . . |
| MCQ300C | Including living and deceased, were any of {SP's/your} |
| | close biological, that is, blood relatives including father, |
| | mother, sisters, or brothers, ever told by a health |
| | professional that they had diabetes? |
| WHD050 | How much did {you/SP} weigh a year ago? |
| WHD110 | How much did {you/SP} weigh 10 years ago? [If you don't |
| | know {your/his/her} exact weight, please make your best guess.] |
| WHD120 | How much did {you/SP} weigh at age 25? [If you don't |
| | know {your/his/her} exact weight, please make your best |
| | guess.] If (you were/she was) pregnant, how much did |
| | (you/she) weigh before (your/her) pregnancy? |
| WHD140 | Up to the present time, what is the most {you have/SP has} |
| | ever weighed? |
| RXDCOUNT | The number of prescription medicines reported |
| DBD910 | During the past 30 days, how often did {you/SP} eat frozen |
| | meals or frozen pizzas? Here are some examples of |
| | frozen meals and frozen pizzas: pepperoni, RED BARON, |
| | veggie, BANQUET classic Salisbury steak meal, etc. |
| FSQ165 | The next questions are about the Food Stamp Program. |
| | Food stamps are usually provided on an electronic debit |
| | card {or EBT card} {called the {{STATE NAME FOR EBT |
| | CARD}} card in {{STATE}}}. Have {you/you or anyone in |
| | your household} ever received Food Stamp benefits? |
| DMDHHSIZ | Total number of people in the household |
| hhIncome | Annual Household Income |
| redFatCal | Has a doctor ever told {you/him/her} to eat less fat to |
| | reduce calories? |
| mostDelta | The difference between the most {you have/he/she has} |
| | ever weighed and {your/his/her} current weight |
| mostBMI | BMI value when {you/he/she} weighed the most |
| tenDelta | Difference between what {you/he/she} weighed 10 years |
| | ago and now |
| tenBMI | BMI value 10 years ago |
| oneBMI | Difference between current BMI and BMI one year ago |
| mostOneDelta | Difference between the most {you have/he/she has} ever |
| | weighed and how much {you/he/she} weighed 1 year ago |
| tenOneDelta | Difference between what {you have/he/she has} weighed |
| | 10 years ago, and how much {you/he/she} weighed 1 year ago |
| probability | Internally generated output from the model based on the |
| | row of answers |

Table 2 illustrates a table that provides descriptions for the column headings of Table 1. The “name” column contains all the column heading symbols of the table from Table 1. The “description” column provides descriptions of the questions referred to by the column heading symbols. For example, DRQSDIET refers to the question “Are you currently on any kind of diet, either to lose weight or for some other health-related reason?”

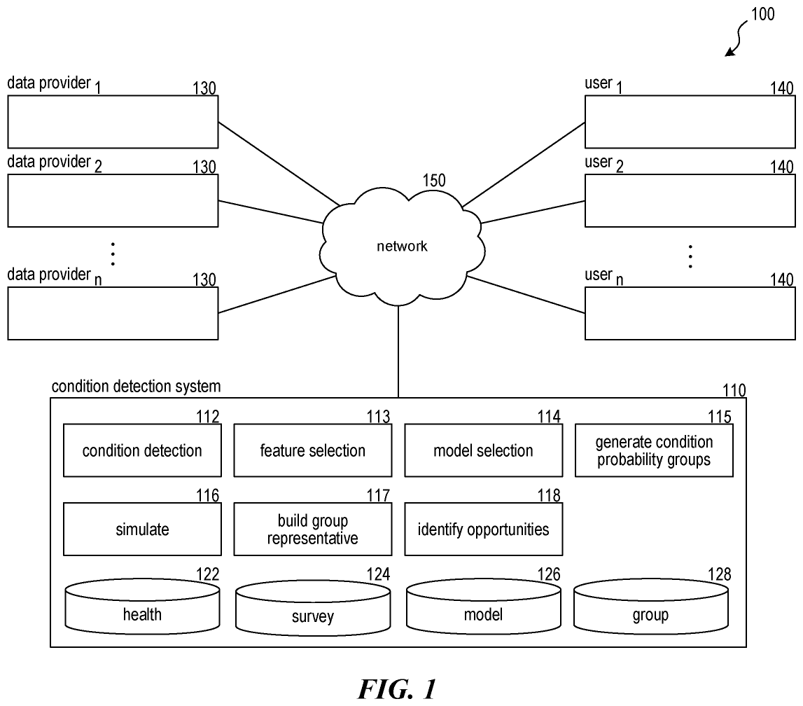

FIG. 1 is a block diagram illustrating an environment 100 in which the condition detection system operates in accordance with some embodiments of the disclosed technology. In this example, environment 100 includes condition detection computing system 110, data provider computing systems 130, user computing systems 140, and network 150. Condition detection computing system 110 comprises condition detection component 112, feature selection component 113, model selection component 114, generate condition probability groups component 115, simulate component 116, build group representative component 117, identify opportunities component 118, health records store 122, survey store 124, model store 126, and condition probability group store 128. Condition detection component 112 is invoked by the condition detection system to detect a condition (probability) within a patient based on input (e.g., survey answers from the patient) and corresponding condition detection models and to identify opportunities for altering the probabilities. Feature selection component 113 is invoked by the condition detection component to identify predictive variable sets (or feature sets). Model selection component 114 is invoked by the condition detection component to select and train condition detection models. Generate condition probability groups component 115 is invoked by the condition detection component to split combinations of survey answers (e.g., any flex answers and non-flex answers) into different groups based on their probabilities for having a corresponding condition. Simulate component 116 is invoked by the generate condition probability groups component to simulate different answer combinations to survey questions for a patient and generate a probability of having a corresponding condition for each combination. Build group representative component 117 is invoked by the generate condition probability groups component to determine average values for survey answers in each of a plurality of condition probability groups. Identify opportunities component 118 in invoked by the condition detection component to identify one or more opportunities for a patient or healthcare provider to take in order to adjust a patient's probability for having or acquiring a particular condition. Health records store 122 stores health records for any number of individuals, such as health records collected from different sources (e.g., data collected from NHANES, BRFSS, the Center for Disease Control, the European Centre for Disease Prevention and Control). Survey store 124 stores information related to surveys, such as survey questions from survey providers and survey answers for one or more survey takers (e.g., patients) for one or more surveys. Thus, the survey store can be used to generate a patient's baseline for any past or current period for which survey answers are stored by retrieving patient data (e.g., survey answers) from the survey store and applying condition detection models to the retrieved data. Moreover, the survey store may also maintain, for each question, a Boolean flag or other indication of whether the question is a flex question or a non-flex question. Model store 126 stores information for each of a plurality of models, such as feature sets, weights, training data, training dates, and so on. Condition probability group store 128 maintains, for each of a plurality of condition probability groups, an indication of corresponding survey questions, answers, a condition probability, a patient identifier, and so on.

One of ordinary skill in the art will recognize that it is not uncommon for information to be generated, retrieved, and/or stored in disparate or non-standard formats. For example, health records or survey results collected from different sources may use different formats. It can be difficult to create a comprehensive view of the collected health records and survey results without processing and storing this information in a standardized form. Thus, in some embodiments, the condition detection system may convert the non-standardized information into a standardized format using, for example, a content server, and store the standardized information in a collection of records in the standardized format. For example, users with remote access to update patient information (e.g., provide new survey results) may provide an update remotely via a network to update information about a patient in the collection of health records in real time through a graphical user interface. In some cases, this update may be in a non-standardized format dependent on the hardware and software platform used by the user. Accordingly, the condition detection system can convert the non-standardized updated information into the standardized format and store the standardized updated information about the patient in the collection of health records in the standardized format. Moreover, the condition detection system can automatically generate a message containing the updated information about the patient, via a content server, whenever updated information has been stored and transmit the message to any one or more of the users (e.g., the patient and other users associated with providing care or treatment to the user) over the network in real time, so that each user has immediate access to up-to-date patient information. The message may include, for example, an updated baseline probability for the user for one or more conditions, an updated list of opportunities for changing the probabilities, and so on. Similarly, the condition detection system may provide real-time updates in response to updating or re-training one or more condition detection models after receiving updated health records, such as an update to the BRFSS database.

Data providers, such as survey providers or other entities that collect and store health data, can interact with the condition detection system via data provider computing systems 130 over network 150 using a user interface provided by, for example, an operating system, web browser, or other application. Users, such as patients, survey respondents, healthcare providers, and so on, can interact with the condition detection system via user computing systems 140 over network 150 using a user interface provided by, for example, an operating system, web browser, or other application. In this example, user computing systems 140, data provider computing systems 130, and condition detection computing system 110 can communicate via network 150.

The computing devices and systems on which the condition detection system can be implemented can include a central processing unit, input devices, output devices (e.g., display devices and speakers), storage devices (e.g., memory and disk drives), network interfaces, graphics processing units, accelerometers, cellular radio link interfaces, global positioning system devices, and so on. The input devices can include keyboards, pointing devices, touchscreens, gesture recognition devices (e.g., for air gestures), thermostats, smart devices, head and eye tracking devices, microphones for voice or speech recognition, and so on. The computing devices can include desktop computers, laptops, tablets, e-readers, personal digital assistants, smartphones, gaming devices, servers, and computer systems such as massively parallel systems. The computing devices can each act as a server (e.g., a content server) or client to other servers or client devices. The computing devices can access computer-readable media that include computer-readable storage media and data transmission media. The computer-readable storage media are tangible storage means that do not include transitory, propagating signals. Examples of computer-readable storage media include memory such as primary memory, cache memory, and secondary memory (e.g., CD, DVD, Blu-Ray) and include other storage means. Moreover, data may be stored in any of a number of data structures and data stores, such as a databases, files, lists, emails, distributed data stores, storage clouds, etc. The computer-readable storage media can have recorded upon or can be encoded with computer-executable instructions or logic that implements the condition detection system, such as a component comprising computer-executable instructions stored in one or more memories for execution by one or more processors. In addition, the stored information can be encrypted. The data transmission media are used for transmitting data via transitory, propagating signals or carrier waves (e.g., electromagnetism) via a wired or wireless connection. In addition, the transmitted information can be encrypted. Additionally, the condition detection system may generate hash values (e.g., MD6, SHA-1, SHA-256) for any stored and/or transmitted data. In some cases, the condition detection system can transmit various alerts to a user based on a transmission schedule, such as an alert to inform the user that an opportunity has or has not been met or that one or more changes can alter a patient's condition probability (i.e., the probability that the patient has a corresponding condition). Furthermore, the condition detection system can transmit an alert over a wireless communication channel to a wireless device associated with a remote user or a computer of the remote user based upon a destination address associated with the user and a transmission schedule in order to, for example, periodically send updated condition probabilities and opportunities based on updated patient data and/or training data. In some cases, such an alert can activate an application to cause the alert to display on a remote user computer and to enable a connection via a universal resource locator (URL) to a data source over the internet, for example, when the wireless device is locally connected to the remote user computer and the remote user computer comes online. Various communications links can be used, such as the internet, a local area network, a wide area network, a point-to-point dial-up connection, a cell phone network, and so on for connecting the computing systems and devices to other computing systems and devices to send and/or receive data, such as via the internet or another network and its networking hardware, such as switches, routers, repeaters, electrical cables and optical fibers, light emitters and receivers, radio transmitters and receivers, and the like. While computing systems and devices configured as described above are typically used to support the operation of the condition detection system, those skilled in the art will appreciate that the condition detection system can be implemented using devices of various types and configurations, and having various components. The computing systems may include a secure cryptoprocessor, such as a tamper-resistant and/or tamper-evident cryptoprocessor, as part of a central processing unit for generating and securely storing keys and for encrypting and decrypting data using the keys in order to protect user information and to ensure confidentiality of information.

The condition detection system can be described in the general context of computer-executable instructions, such as program modules and components, executed by one or more computers, processors, or other devices, including single-board computers and on-demand cloud computing platforms. Generally, program modules or components include routines, programs, objects, data structures, and so on that perform particular tasks or implement particular data types. Typically, the functionality of the program modules can be combined or distributed as desired in various embodiments. Aspects of the condition detection system can be implemented in hardware using, for example, an application-specific integrated circuit (“ASIC”) or field programmable gate array (“FPGA”).

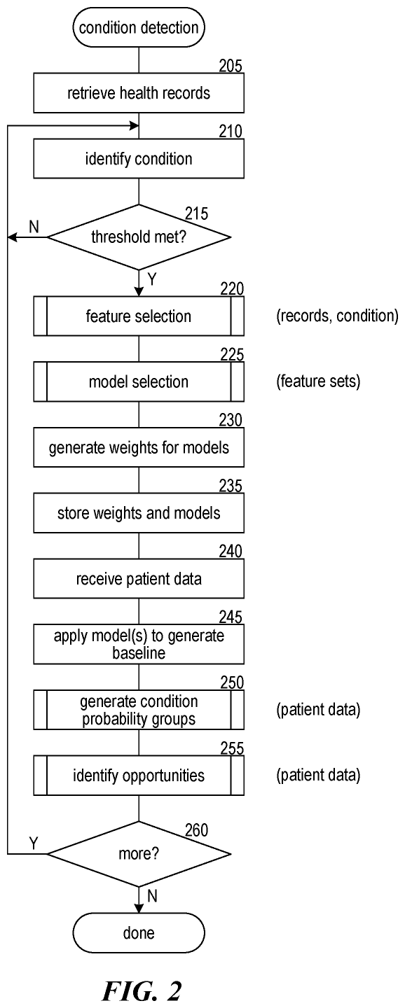

FIG. 2 is a flow diagram illustrating the processing of a condition detection component in accordance with some embodiments of the disclosed technology. In this example, the condition detection system invokes the condition detection component 112 to detect a condition (probability) within a patient (or of the patient acquiring the condition) based on input (e.g., survey answers from the patient) and corresponding condition detection models and to identify opportunities for altering the probabilities. In some cases, the component may be invoked in response to a request from a patient or healthcare provider to generate a baseline probability for a particular condition. In block 205, the component retrieves health records from, for example, one or more data providers, a health records store, and so on. In some cases, the component may standardize the retrieved data. In block 210, the component identifies a condition to detect, such as condition identified in a received request, a randomly selected condition, and so on. The identified condition may correspond to a single variable in the health records (e.g., the response to the question “Do you have diabetes?” or an indication of whether a person has been diagnosed with diabetes) or a set of variables indicative of whether of person has a particular condition. In decision block 215, if more than a threshold number of the retrieved health records have an indication of whether the condition is or is not present in the corresponding individual, then processing continues at block 220, else the component loops back to block 210 to identify another condition for detection (e.g., prompting a user to select a new condition for detection or randomly selecting another condition). The threshold may be a fixed number (e.g., 2,000) set by a user, determined dynamically based on the number of records in the retrieved data (e.g., 25%), and so on. In block 220, the component invokes a feature selection component based on the retrieved health records and the identified condition to generate feature sets. In block 225, the component invokes a model selection component based on the feature sets generated by the feature selection component. In block 230, the component generates weights for the selected model(s) using, for example, a hyper parameterization process (e.g., Bayesian optimization, gradient-based optimization, grid search, random search). In block 235, the component stores the model(s) and corresponding weights in, for example, a model store. In block 240, the component retrieves patient data, such as the patient's responses to one or more survey questions. In block 245, the component applies the models to the retrieved patient data to generate a new or current baseline for the patient. In block 250, the component invokes a generate condition probability groups component based on the retrieved patient data. In block 255, the component invokes an identify opportunities component. In decision block 260, if there are additional conditions to detect, then the component loops back to block 210 to identify another condition to detect, else processing of the component completes. Thus, the condition detection component can be invoked to generate models and apply them to patient data for a plurality of different conditions. In some embodiments, a means for detecting a condition comprises one or more computers or processors configured to carry out an algorithm disclosed in FIG. 2 and this paragraph.

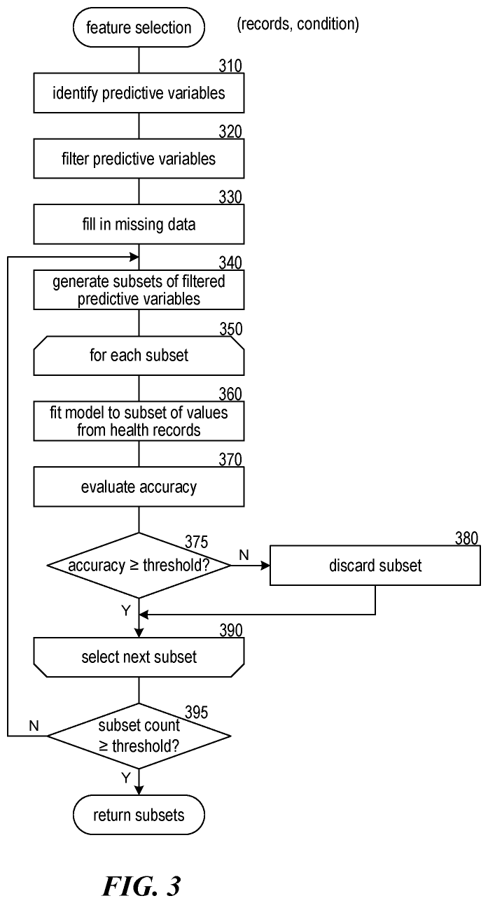

FIG. 3 is a block diagram illustrating the processing of a feature selection component in accordance with some embodiments of the disclosed technology. In this example, the condition detection component invokes the feature selection component to identify predictive variable sets (or feature sets). In block 310, the component identifies predictive variables from among the variables represented in the health records based on the ability of each variable to predict the condition for an individual. For example, the component may generate a predictive score for each variable relative to the condition, determine a correlation score between each variable and the condition, determine a predictive power score for each variable relative to the condition, and so on. In block 320, the component filters out variables, such as variables that are closely related to the condition, variables deemed to be spurious by a user, variables otherwise identified by a user for removal, and so on. In block 330, the component fills in missing data for the filtered (remaining) predictive variables by, for example, calculating an aggregate (e.g., average) value for a corresponding variable based on data in the health records for that variable. In block 340, the component generates subsets of the filtered (remaining) predictive variables. In some cases, the component generates subsets by randomly selecting a predetermined number of predictive variables from the filtered (remaining) predictive variables. In other cases, the component generates every combination of a predetermined number or percentage of the filtered (remaining) predictive variables. In some cases, the component receives, from a user, an indication of subsets of the filtered (remaining) predictive variables to use as a basis for the generating. It will be appreciated that each subset represents a set of questions that may be presented to a user as part of a survey to be used by the condition detection system. Accordingly, the condition detection can cap the number of predictive variables in a subset to avoid producing a survey that patients may find overwhelming, although in some cases such a survey may be appropriate. In blocks 350-390, the component loops through each of the generated subsets (and the underlying values from the health records) to assess the accuracy of each subset, with each subset corresponding to a feature set that may be selected for use in training one or more machine learning models. In block 360, the component fits a model, such as a generalized linear model, to the values of the subset of predictive variables from the health records. In block 370, the component evaluates the accuracy of the subset based on, for example, the type I and type II errors generated when fitting the model to the subset of variables, such as a count of the type I and type II errors, a ratio between the type I and type II errors, and so on. In block 375, if the accuracy of the subset is greater than an accuracy threshold, the component continues at block 390, else the component continues at block 380. The accuracy threshold may be determined by a user or automatically by the condition detection system based on previous tests, such as within 15% of the most accurate subset tested thus far. In block 380, the component discards the subset as not being accurate enough. In block 390, if there are additional subsets to select, the component selects the next subset and loops back to block 350, else the component continues at block 395. One of ordinary skill in the art will recognize that subsets may be selected by first determining the accuracy of each and then selecting a predetermined number or percentage of the most accurate subsets (e.g., top 10, top 20, top 3%, top 25%) rather than making a determination before processing all of the subsets. In decision block 395, if the number of subsets remaining (i.e., those that were not discarded) exceeds a count threshold, the component returns the remaining subsets as feature sets, else the component loops back to block 340 to generate subsets of filtered predictive variables. In some cases, rather than returning multiple subsets as feature sets, the component returns the union of the subsets as a single feature set if, for example, the union is less than a predetermined number of variables. In some embodiments, a means for selecting features comprises one or more computers or processors configured to carry out an algorithm disclosed in FIG. 3 and this paragraph.

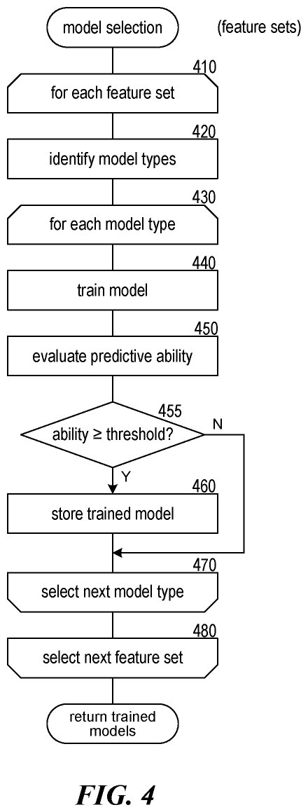

FIG. 4 is a flow diagram illustrating the processing of a model selection component in accordance with some embodiments of the disclosed technology. In this example, the condition detection component invokes the model selection component to select and train machine learning models (for generating condition probabilities) based on generated feature sets. In some cases, the condition detection system may skip the training and simply select previously trained models from a model store, such as recently trained models, rather than training new models. In blocks 410-480, the component loops through each of the feature sets to train one or more machine learning models using the feature set and to assess the accuracy of the trained machine learning model(s). In block 420, the component identifies one or more machine learning model types that are to be trained using the feature set, such as a set of machine learning model types identified by a user, a randomly selected set of available machine learning model types, and so on. For example, the machine learning model types may be any type of classification model (or classifier) such as Gaussian models, boosting models, neural networks (e.g., fully connected, convolutional, recurrent, autoencoder, or restricted Boltzmann machine), support vector machines, Bayesian classifiers, k-means classifiers, and so on. In blocks 430-470, the component loops through each of the identified machine learning model types to train one or more models using the feature set and to assess the accuracy of the trained model(s). In block 440, the component applies machine learning techniques to train a model of the currently selected model type using the health record data as training data. One of ordinary skill in the art will recognize that training the models can include identifying a portion of the health records as training data and another portion as validation data, and identifying variables as independent (the feature sets) and dependent (the variable(s) corresponding to the condition to be detected by the model). In block 450, the component evaluates the predictive ability of the trained model based on, for example, an analysis of type I and type II errors. In block 455, if the predictive ability of the trained model is greater than or equal to a model accuracy threshold, then the component continues at block 460, else the component continues at block 470. In block 460, the component stores the trained model including, for example, a label for the model, a date/time at which the model was trained, an indication of the training data, an indication of the model's independent and dependent variables, and so on. In block 470, if there are additional model types to be selected, the component selects the next model type and loops back to block 430, else the component continues at block 480. In block 480, if there are additional feature sets to be selected, the component selects the next feature set and loops back to block 410, else the component returns the trained model(s). In some embodiments, a means for selecting models comprises one or more computers or processors configured to carry out an algorithm disclosed in FIG. 4 and this paragraph.

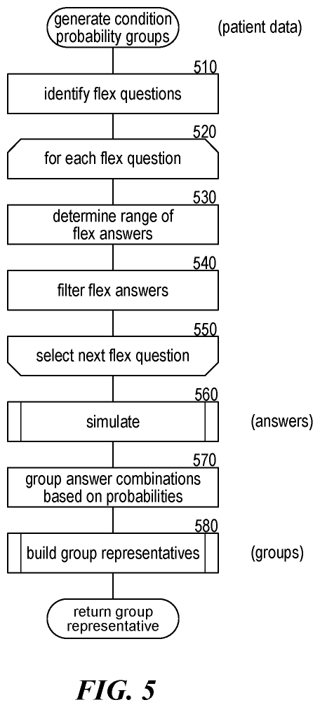

FIG. 5 is a flow diagram illustrating the processing of a generate condition probability groups component in accordance with some embodiments of the disclosed technology. In this example, the condition detection component invokes the generate condition probability groups component to generate flex answers based on patient data (e.g., answers to a survey received from a patient) and split combinations of survey answers (flex answers and non-flex answers) into different groups based on their condition probabilities. In block 510, the component identifies flex questions from the patient data by, for example, identifying corresponding flags in the patient data, a corresponding record in a survey data store, and so on. In blocks 520-550, the component loops through each of the flex questions and expands (or “flexes”) each into a set of hypothetical answers for the patient. In block 530, the component determines a range of flex answers for the flex question. For example, the component may analyze survey or health records for a corresponding variable and identify every answer or value that has been provided for the corresponding variable, such as every answer received to the question “What is your waist size?,” or value logged for a corresponding variable. As another example, the component may generate every possible value based on MIN and MAX values associated with the variable (e.g., metadata stored in a survey store) and a corresponding level of precision (e.g., ones, tenths, hundredths, thousandths). In block 540, the component filters the answers by, for example, setting lower and upper limits on the values for flex answers based on the patient's answer to the question and associated data. For example, for continuous variables, the component may set a minimum flex answer as a predetermined percentage (e.g., 50%, 66%, 75%) of the patient's answer to the corresponding question and a maximum flex answer as a predetermined percentage (e.g., 110%, 150%, 200%) of the patient's answer. As another example, the component may use the patient's height as a basis for filtering potential weight answers from the determination by identifying only weight values for individuals within a range (e.g., +/−20%) of the patient's height. In this manner, the component can conserve valuable computing resources when generating the probability groups. In block 550, if there are additional flex questions to be selected, the component selects the flex question and loops back to block 520, else the component continues at block 560. In block 560, the component invokes a simulate component to generate condition probabilities for each combination of flex answers and non-flex answers for the patient. In block 570, the component groups answer combinations based on their corresponding probabilities. For example, the component may generate a number of equally sized groups (e.g., quartiles, deciles, five groups, 99 groups), may group the combinations into potentially uneven groups based on their probabilities (e.g., 0.0-0.1, 0.1-0.2, 0.2-0.3, 0.3-0.5, 0.5-0.7, 0.7-1.0), and so on. In block 580, the component invokes a build group representative component to generate average values for each group and then completes. In some embodiments, a means for generating condition probability groups comprises one or more computers or processors configured to carry out an algorithm disclosed in FIG. 5 and this paragraph.

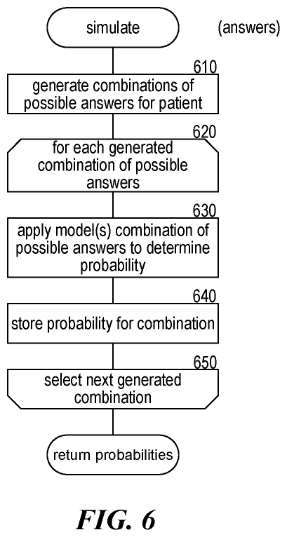

FIG. 6 is a flow diagram illustrating the processing of a simulate component in accordance with some embodiments of the disclosed technology. In this example, the generate condition probability group component invokes the simulate component to simulate different answer combinations to a set of survey questions for a patient and generate a condition probability for each combination of survey answers. Thus, the component simulates the different combinations of answers that the patient may provide to the set of survey questions based on flex answers and non-flex answers. In block 610, the component generates combinations of possible answers to the set of survey questions for the patient. Thus, the number of combinations corresponds to the product of the counts of flex answers for each flex question (there being no more than one non-flex answer to any non-flex question). In other words, if a survey has 10 flex questions, where five of the flex questions each have 10 potential flex answers and the other five flex questions each have five potential flex answers, the number of combinations would be 31,250,000 (10×10×10×10×10×5×5×5×5×5). In blocks 620-650, the component loops through each of the combinations to generate a condition probability for the combination. In block 630, the component applies the condition detection model trained for detecting the probability of having the condition to the currently selected combination to produce or generate a condition probability. In block 640, the component stores the probability in association with the combination. In block 650, if there are additional generated combinations to be selected, the component selects the next generated combination and loops back to block 620, else the component returns the determined probabilities. In some embodiments, a means for simulating survey answers comprises one or more computers or processors configured to carry out an algorithm disclosed in FIG. 6 and this paragraph.

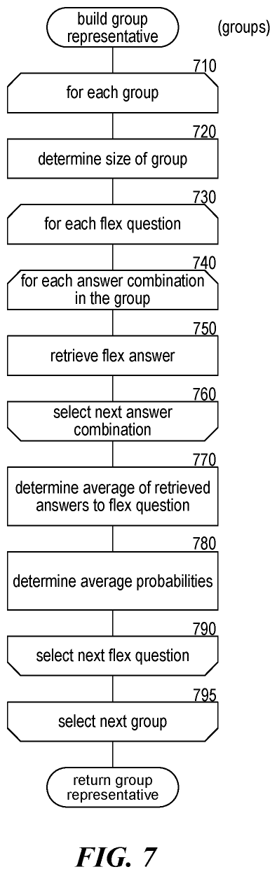

FIG. 7 is a flow diagram illustrating the processing of a build group representative component in accordance with some embodiments of the disclosed technology. In this example, the generate condition probability component invokes the build group representative component to determine aggregate values for survey answers in each of a number of condition probability groups. In blocks 710-790, the component loops through each condition probability group to build a representative set of values for each of the variables. In block 720, the component determines the size of the condition probability group (i.e., the number of combinations of answers represented by the condition probability group). In blocks 730-760, the component loops through each flex question to determine an aggregate value for flex answers to the question for the condition probability group, such as an average value. In blocks 740-760, the component loops through each answer combination in the condition probability group. In block 750, the component retrieves the flex answer to the flex question in the current answer combination. In block 760, if there are additional answer combinations to be selected, the component selects the next answer combination and loops back to block 740, else the component continues at block 770. In block 770, the component determines an average flex answer for the current flex question and the current condition probability group, such as the mean, median, or mode, based on the retrieved flex answers. In block 780, the component determines a condition probability value for the current condition probability group based on the condition probabilities determined for each answer combination in the condition probability group, such as the mean, median, or mode. In some cases, the component may use a different technique for determining a probability value for each condition probability group, such as using a maximum or minimum condition probability in the condition probability group or by applying the trained condition detection model to the non-flex answers and the average values for the flex answers in the condition probability group, and so on. In block 790, if there are additional flex questions to be selected, the component selects the next flex question and loops back to block 730, else the component continues at block 795. In block 795, if there are additional condition probability groups to be selected, the component selects the next condition probability group and loops back to block 710, else processing of the component completes. In some embodiments, a means for building group representatives comprises one or more computers or processors configured to carry out an algorithm disclosed in FIG. 7 and this paragraph.

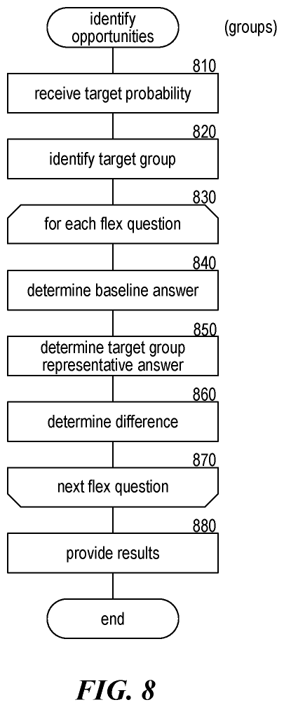

FIG. 8 is a flow diagram illustrating the processing of an identify opportunities component in accordance with some embodiments of the disclosed technology. In this example, the condition detection component invokes the identify opportunities component to identify one or more opportunities for a patient or healthcare provider to take in order to adjust a patient's probability for having or acquiring a particular condition. In block 810, the component receives a target probability for the patient, such as a specific target received from the patient or an amount by which the patient would like to change their condition probability. In block 820, the component identifies a target group from among the condition probabilities by comparing the target probability to the average probabilities associated with each group. For example, the component may identify the group with the condition probability that is nearest the target probability, the group with the nearest condition probability that is also less than the target probability, the group with the nearest condition probability that is also greater than the target probability, and so on. In blocks 830-870, the component loops through each flex question to compare the target group's representative values for each flex question and to compare those values to the patient's baseline. In block 840, the component determines the patient's baseline answer for the current flex question based on the patient's survey answers. In block 850, the component determines the target group's representative's value for the flex question by, for example, retrieving the value from a group store. In block 860, the component determines the difference between the patient's baseline answer and the target group's representative's value for the flex question. In block 870, if there are additional flex questions to be selected, the component selects the next flex question and loops back to block 830, else the component continues at block 880. In block 880, the component provides results of the comparison by, for example, displaying a table or chart that includes, for each flex question, an indication of the determined differences. In some embodiments, a means for identifying opportunities comprises one or more computers or processors configured to carry out an algorithm disclosed in FIG. 8 and this paragraph.

The above Detailed Description of examples of the disclosed subject matter is not intended to be exhaustive or to limit the disclosed subject matter to the precise form disclosed above. While specific examples for the disclosed subject matter are described above for illustrative purposes, various equivalent modifications are possible within the scope of the disclosed subject matter, as those skilled in the relevant art will recognize. For example, while processes or blocks are presented in a given order, alternative implementations can perform routines having steps, or employ systems having blocks, in a different order, and some processes or blocks can be deleted, moved, added, subdivided, combined, and/or modified to provide alternative combinations or sub-combinations. Each of these processes or blocks can be implemented in a variety of different ways. Also, while processes or blocks are at times shown as being performed in series, these processes or blocks can instead be performed or implemented in parallel, or can be performed at different times and/or in different orders, shown steps may be omitted, or other steps included. Further, any specific numbers noted herein are only examples: alternative implementations can employ differing values or ranges.

The disclosure provided herein can be applied to other systems and is not limited to the system described herein. The features and acts of various examples included herein can be combined to provide further implementations of the disclosed subject matter. Some alternative implementations of the disclosed subject matter can include not only additional elements to those implementations noted above, but also can include fewer elements.

Any patents, applications, and other references noted herein are incorporated herein by reference in their entireties. Aspects of the disclosed subject matter can be changed, if necessary, to employ the systems, functions, components, and concepts of the various references described herein to provide yet further implementations of the disclosed subject matter.

These and other changes can be made in light of the above Detailed Description. While the above disclosure includes certain examples of the disclosed subject matter, along with the best mode contemplated, the disclosed subject matter can be practiced in any number of ways. Details of the condition detection system can vary considerably in the specific implementation, while still being encompassed by this disclosure. Terminology used when describing certain features or aspects of the disclosed subject matter does not imply that the terminology is being redefined herein to be restricted to any specific characteristics, features, or aspects of the disclosed subject matter with which that terminology is associated. The scope of the disclosed subject matter encompasses not only the disclosed examples, but also all equivalent ways of practicing or implementing the disclosed subject matter under the claims.

Although the subject matter has been described in language specific to structural features and/or methodological acts, it is to be understood that the subject matter defined in the appended claims is not necessarily limited to the specific features or acts described above. The specific features and acts described above are disclosed as example forms of implementing the claims.

The following paragraphs describe various embodiments of aspects of the condition detection system. An implementation of the condition detection system may employ any combination of the embodiments. The processing described below may be performed by a computing system with a processor that executes computer-executable instructions stored on a computer readable storage medium that implements the condition detection system.

In some embodiments, a method, performed by a computing system having one or more processors, for determining a condition probability is provided. In some embodiments, the method receives, from one or more sources, health records for a plurality of corresponding individuals. The received health records comprising a value for each of a plurality of variables. In some embodiments, the method identifies a condition for which to generate a condition probability for a patient. In some embodiments, the method identifies, from among the received health records, health records that include an indication of whether the corresponding individual has the identified condition. In some embodiments, the method selects feature sets based at least in part on the plurality of variables from the received health records and selects models based at least in part on the selected feature sets. In some embodiments, the method generates weights for the selected models. In some embodiments, the method receives data for the patient. In some embodiments, the method applies the selected models to the received data for the patient to generate a probability of the patient having the identified condition. In some embodiments, the method selects features by, for each of a plurality of subsets of variables of the plurality of variables, fitting a model to the subset of variables to determine an accuracy for the subset of variables, comparing the determined accuracy to a first threshold, and response to determining that the determined accuracy is greater than or equal to the first threshold, selecting the subset of variables as a feature set. In some embodiments, the method selects models by for each of the plurality of features sets, for each of a plurality of model types: training a model of the model type based on the feature set and at least a portion of the received health records, evaluating the predictive ability of the trained model, comparing the predictive ability of the trained model to a second threshold, and in response to determining that the predictive ability of the trained model is greater than or equal to the second threshold, selecting and storing the trained model. The received data may be received, from the patient, answers to each of a plurality of survey questions. In some embodiments, the method generates condition probability groups for the patient. In some embodiments, the method receives, from the patient, an indication of a desired probability. In some embodiments, the method identifies one or more opportunities based at least in part on the desired probability and the generated condition probability groups. In some embodiments, the method generates condition probability groups for the patient comprises by identifying a plurality of flex questions, for each of the identified plurality of flex questions, determining a plurality of flex answers for the flex question; generating combinations of answers based on the received data for the patient, wherein the answers include flex answers and non-flex answers; for each generated combination of answers, applying a condition detection model to the combination to generate a condition probability for the combination; and grouping the combinations into a plurality of groups based on the generated condition probabilities. In some embodiments, the method identifies one or more opportunities based at least in part on the desired probabilities and the generated condition probability groups by receiving, from the patient, a target probability and identifying one of the plurality of groups corresponding to the target probability. In some embodiments, the method, for each of a plurality of condition probability groups, generating aggregates values forflex answers in the condition probability group, and builds a group representative based at least in part on aggregate values generated for the condition probability group. In some embodiments, the method applies one or more transformations to each of a plurality of the received health records to create a modified set of health records. In some embodiments, the method creates a first training set comprising the plurality of the received health records and the modified set of health records. In some embodiments, the method trains a neural network in a first stage of training using the first training set. In some embodiments, the method creates a second training set for a second stage of training comprising the first training set and records for individuals that are incorrectly detected as having the identified condition after the first stage of training. In some embodiments, the method trains the neural network in a second stage using the second training set.

In some embodiments, a computer-readable storage medium storing instructions that, when executed by a computing system having at least one processor and at least one memory, cause the computing system to perform a method for determining condition probabilities is provided. In some embodiments, the method receives records of variables of individuals. In some embodiments, the method receives a selection of a variable set of variables. In some embodiments, the method generates a predictive score for each variable of the variable set to identify predictive variables. In some embodiments, the method fits a generalized linear model to subsets of the identified predictive variables to determine a predictive capability of each subset. In some embodiments, the method eliminates predictive variables without sufficient predictive capability. In some embodiments, the method identifies one or more models based on an analysis of the predictive accuracy of combinations of models. In some embodiments, the method generates a weight for each model. In some embodiments, the method, in response to identifying one or more models based on analysis of the predictive accuracy of combinations of models, trains the identified one or more models based on at least a portion of the received records. In some embodiments, the method further receives patient data and applies one or more trained models to the received patient data. In some embodiments, the method generating the predictive score for a first variable of the variable set comprises: identifying one or more demographic variables, identifying one or more records from among the received records that include values for the identified one or more demographic variables, applying a first model to the one or more demographic variables and corresponding values to determine a first predictive value, appending values for the first variable to the values for the demographic variables to create composite data, applying the first model to the composite data to determine a second predictive value, and comparing the first predictive value to the second predictive value to determine an information gain for the first variable. In some embodiments, the method eliminates a first subset of predictive variables without sufficient predictive capability identifying type I errors generated when fitting the fitting the generalized linear model to the first subset of predictive variables and/or identifying type II errors generated when fitting the fitting the generalized linear model to the first subset of predictive variables. In some embodiments, the method further receives patient data, generates combinations of answers based on the received patient data, wherein the answers include flex answers and non-flex answers. In some embodiments, the method, for each generated combination of answers, applies a condition detection model to the combination to generate a condition probability for the combination. In some embodiments, the method groups the combinations into a plurality of groups based on the generated condition probabilities. In some embodiments, the method, for each of the plurality of groups, for each of a plurality of flex questions, generates aggregate values for flex answers associated with the question and the group.

In some embodiments, the method a computing system for determining condition probabilities is provided. In some embodiments, the computing system comprises at least one memory and/or at least one processor. In some embodiments, the computing system comprises a component configured to receive, from one or more sources, records for a plurality of corresponding individuals, the records comprising a value for each of a plurality of variables. In some embodiments, the computing system comprises a component configured to identify a condition for which to generate a condition probability for a user. In some embodiments, the computing system comprises a component configured to identify, from among the received records, records that include an indication of whether the corresponding individual has the identified condition. In some embodiments, the computing system comprises a component configured to select feature sets based at least in part on the plurality of variables from the received records. In some embodiments, the computing system comprises a component configured to select models based at least in part on the selected feature sets. In some embodiments, the computing system comprises a component configured to apply the selected models to received data for the user to generate a probability of the user having the identified condition. In some embodiments, each component of the computing system comprises computer-executable instructions stored in the at least one memory for execution by the at least one processor. In some embodiments, the received records are health records and wherein the condition is a disease, disorder, or syndrome. In some embodiments, the computing system comprises a component configured to present a survey to the user. In some embodiments, the computing system comprises a component configured to receive the received data from the user via the presented survey. In some embodiments, the computing system comprises a survey store storing a plurality of records, each corresponding to one or more survey questions, wherein the survey store comprises, for each of the one or more survey questions, an indication of whether the survey question is a flex question. In some embodiments, the computing system comprises a component configured to generate a baseline for the user, the baseline for the user comprising a baseline value for each of a plurality of variables. In some embodiments, the computing system comprises a component configured to receive a target condition probability for the user. In some embodiments, the computing system comprises a component configured to identify a target condition probability group based at least in part on the target condition probability for the user, the condition probability group comprising a target value for each of the plurality of variables. In some embodiments, the computing system comprises a component configured to, for each of the plurality of variables, compare the baseline value for the variable to the target value for the variable.

From the foregoing, it will be appreciated that specific embodiments of the disclosed subject matter have been described herein for purposes of illustration, but that various modifications can be made without deviating from the scope of the disclosed subject matter. For example, while diseases have been described as one type of condition, one of ordinary skill in the art will recognize that any condition (malignant, benign, etc.) may be detected by the condition detection system. Moreover, while various conditions have been used as examples herein, one of ordinary skill in the art will recognize that the condition detection system can be used to detect any type of condition, such as the condition of homes, automobiles, organizations, and so on. For example, attributes of automobiles may be maintained over time by their owners and/or service technicians/agencies. These attributes can be used to build a set of training data that can be used to train models that predict conditions within automobiles, such as a faulty or blown head gasket, worn brakes, engine issues, and so on. These models can be applied to the current condition of an automobile to detect conditions within the vehicle and identify opportunities to take pre-emptive steps to maintain the automobile. Furthermore, in some cases the condition detection system may also provide a survey creation form or dialog for users to customize surveys and associated questions or may automatically generate surveys based on one or more generated features sets by, for example, populating a survey data structure with questions corresponding to each feature in one or more feature sets. As another example, in some cases, the condition probability system stores health records in a standardized format about a patient in a plurality of network-based non-transitory storage devices having a collection of health records stored thereon, provides remote access to users over a network so any one of the users can update the information about the patient in the collection of medical records in real time through a graphical user interface, wherein at least one of the users provides the updated information in a non-standardized format dependent on the hardware and software platform used by the at least one user, wherein the users comprise the patient and at least one health care provider associated with the patient, converts, by a content server, the non-standardized updated information into the standardized format, stores the standardized updated information about the patient in the collection of medical records in the standardized format, generates an updated condition probability for the patient based at least in part on the updated information about the patient, automatically generates a message containing an indication of the updated condition probability by the content server whenever a stored condition probability is updated, and transmits the message to all of the users over the computer network in real time, so that each user has immediate access to up-to-date patient information regarding the updated condition probability. In some examples, the condition detection system uses classifiers, such as a neural network, to classify targets or users as either having a particular condition (or conditions) or not, based upon the training of one or more models on a set of records (e.g., health records) of individuals that do and do not have the condition (or conditions). In some examples, the condition detection model(s) is trained using collected data (e.g., health records) along with transformed versions of the underlying collected data using, for example, stochastic learning with backpropagation (SLBP) to adjust the weights of a neural network. In some cases, the use of this augmented training set may increase type I and/or type II errors while classifying. The condition detection system can reduce these errors by performing an iterative training algorithm, in which the condition detection model(s) is retrained with an updated training set containing the incorrectly classified records after condition detection has been performed (i.e., the records or transformed versions of those records for which a condition was incorrectly detected), which provides a condition detection model that can detect condition(s) (probabilities) in the underlying data while limiting the number of type I and/or type II errors. In order to manage a patient's health, it is important to periodically determine where the patient is on a probability scale without respect to having any number of conditions. A number of techniques are disclosed for helping the patient and medical workers in handling their shared responsibilities, including techniques for detecting condition probabilities and identified opportunities to create and/or modify patient care plans based on these condition probabilities. Additionally, while advantages associated with certain embodiments of the new technology have been described in the context of those embodiments, other embodiments can also exhibit such advantages, and not all embodiments need necessarily exhibit such advantages to fall within the scope of the technology. Accordingly, the disclosure and associated technology can encompass other embodiments not expressly shown or described herein. Although the subject matter has been described in language specific to structural features and/or methodological acts, it is to be understood that the disclosed subject matter is not necessarily limited to the specific features or acts described above. Rather, the specific features and acts described above are disclosed as example forms of the disclosed subject matter. To the extent any materials incorporated herein by reference conflict with the present disclosure, the present disclosure controls.

I/We claim:

1. A method, performed by a computing system having one or more processors, for determining a condition probability, the method comprising:
receiving, from one or more sources, health records for a plurality of corresponding individuals, the received health records comprising a value for each of a plurality of variables;
identifying a condition for which to generate a condition probability for a patient;
identifying, from among the received health records, health records that include an indication of whether the corresponding individual has the identified condition;
selecting feature sets based at least in part on the plurality of variables from the received health records;
selecting models based at least in part on the selected feature sets;
generating weights for the selected models;
receiving data for the patient; and
applying the selected models to the received data for the patient to generate a probability of the patient having the identified condition.

2. The method of claim 1, wherein selecting features comprises:
for each of a plurality of subsets of variables of the plurality of variables,
fitting a model to the subset of variables to determine an accuracy for the subset of variables,
comparing the determined accuracy to a first threshold, and
in response to determining that the determined accuracy is greater than or equal to the first threshold, selecting the subset of variables as a feature set.

3. The method of claim 2, wherein selecting models comprises:
for each of the plurality of features sets,
for each of a plurality of model types,
training a model of the model type based on the feature set and at least a portion of the received health records,
evaluating the predictive ability of the trained model,
comparing the predictive ability of the trained model to a second threshold, and
in response to determining that the predictive ability of the trained model is greater than or equal to the second threshold, selecting and storing the trained model.

4. The method of claim 1, wherein receiving data for the patient comprises receiving, from the patient, answers to each of a plurality of survey questions.

5. The method of claim 1, further comprising:
generating condition probability groups for the patient;
receiving, from the patient, an indication of a desired probability; and
identifying one or more opportunities based at least in part on the desired probability and the generated condition probability groups.

6. The method of claim 5, wherein generating condition probability groups for the patient comprises:
identifying a plurality of flex questions;
for each of the identified plurality of flex questions,
determining a plurality of flex answers for the flex question;
generating combinations of answers based on the received data for the patient, wherein the answers include flex answers and non-flex answers;
for each generated combination of answers,
applying a condition detection model to the combination to generate a condition probability for the combination; and
grouping the combinations into a plurality of condition probability groups based on the generated condition probabilities.

7. The method of claim 6, wherein identifying one or more opportunities based at least in part on the desired probabilities and the generated condition probability groups comprises:
receiving, from the patient, a target probability; and
identifying one of the plurality of condition probability groups corresponding to the target probability.

8. The method of claim 6, further comprising:
for each of the plurality of condition probability groups,
generating aggregate values for flex answers in the condition probability group, and
building a group representative based at least in part on aggregate values generated for the condition probability group.

9. The method of claim 1, further comprising:
applying one or more transformations to each of a plurality of the received health records to create a modified set of health records;
creating a first training set comprising the plurality of the received health records and the modified set of health records;
training a neural network in a first stage of training using the first training set;
creating a second training set for a second stage of training comprising the first training set and records for individuals that are incorrectly detected as having the identified condition after the first stage of training; and
training the neural network in a second stage using the second training set.

10. A computer-readable storage medium storing instructions that, when executed by a computing system having at least one processor and at least one memory, cause the computing system to perform a method for determining condition probabilities, the method comprising:
receiving records of variables of individuals;
receiving a selection of a variable set of variables;
generating a predictive score for each variable of the variable set to identify predictive variables;
fitting a generalized linear model to subsets of the identified predictive variables to determine a predictive capability of each subset;
eliminating predictive variables without sufficient predictive capability;
identifying one or more models based on an analysis of the predictive accuracy of combinations of models; and
generating a weight for each model.

11. The computer-readable storage medium of claim 10, the method further comprising:
in response to identifying one or more models based on analysis of the predictive accuracy of combinations of models, training the identified one or more models based on at least a portion of the received records.

12. The computer-readable storage medium of claim 11, the method further comprising:
receiving patient data; and
applying the one or more trained models to the received patient data.

13. The computer-readable storage medium of claim 10, wherein generating the predictive score for a first variable of the variable set comprises:
identifying one or more demographic variables;
identifying one or more records from among the received records that include values for the identified one or more demographic variables;
applying a first model to the one or more demographic variables and corresponding values to determine a first predictive value;
appending values for the first variable to the values for the demographic variables to create composite data;
applying the first model to the composite data to determine a second predictive value; and
comparing the first predictive value to the second predictive value to determine an information gain for the first variable.

14. The computer-readable storage medium of claim 10, wherein eliminating a first subset of predictive variables without sufficient predictive capability comprises:
identifying type I errors generated when fitting the fitting the generalized linear model to the first subset of predictive variables; and
identifying type II errors generated when fitting the fitting the generalized linear model to the first subset of predictive variables.

15. The computer-readable storage medium of claim 10, the method further comprising:
receiving patient data;
generating combinations of answers based on the received patient data, wherein the answers include flex answers and non-flex answers;
for each generated combination of answers,
applying a condition detection model to the combination to generate a condition probability for the combination;
grouping the combinations into a plurality of groups based on the generated condition probabilities; and
for each of the plurality of groups,
for each of a plurality of flex questions,
generating aggregate values for flex answers associated with the question and the group.

16. A computing system for determining condition probabilities, the computing system comprising:
at least one memory;
at least one processor;
a component configured to receive, from one or more sources, records for a plurality of corresponding individuals, the records comprising a value for each of a plurality of variables;
a component configured to identify a condition for which to generate a condition probability for a user;
a component configured to identify, from among the received records, records that include an indication of whether the corresponding individual has the identified condition;
a component configured to select feature sets based at least in part on the plurality of variables from the received records;
a component configured to select models based at least in part on the selected feature sets;
a component configured to apply the selected models to received data for the user to generate a probability of the user having the identified condition,
wherein each component comprises computer-executable instructions stored in the at least one memory for execution by the at least one processor.

17. The computing system of claim 16, wherein the received records are health records and wherein the condition is a disease, disorder, or syndrome.

18. The computing system of claim 16, further comprising:
a component configured to present a survey to the user; and
a component configured to receive the received data from the user via the presented survey.

19. The computing system of claim 16, further comprising:
a survey store storing a plurality of records, each corresponding to one or more survey questions, wherein the survey store includes, for each of the one or more survey questions, an indication of whether the survey question is a flex question.

20. The computing system of claim 16, further comprising:
a component configured to generate a baseline for the user, the baseline for the user comprising a baseline value for each of a plurality of variables;
a component configured to receive a target condition probability for the user;
a component configured to identify a target condition probability group based at least in part on the target condition probability for the user, the condition probability group comprising a target value for each of the plurality of variables; and
a component configured to, for each of the plurality of variables, compare the baseline value for the variable to the target value for the variable.
